(19) United States
(12) Patent Application Publication
(10) Pub. No.: US 2023/0419816 A1
(43) Pub. Date: Dec. 28, 2023

(54) MULTI-MEDIA SESSION AND COMMUNICATIONS IN A WIRELESS NETWORK

(71) Applicant: Charter Communications Operating, LLC, St. Louis, MO (US)

(72) Inventor: Ashutosh K. Sinha, Centennial, CO (US)

(21) Appl. No.: 17/846,285

(22) Filed: Jun. 22, 2022

(51) Int. Cl. G08B 25/01; H04W 76/10; G08B 25/10; A61B 5/00; H04W 12/033

(52) U.S. Cl. CPC G08B 25/016; H04W 76/10; G08B 25/10; A61B 5/0002; H04W 12/033; H04W 80/10

(57) ABSTRACT

A network environment includes a mobile communication device (a.k.a., user equipment), a wireless network, and a data management resource. In response to receiving input from the user operating the communication device, via first signaling associated with a communication session protocol, the mobile communication device establishes a voice channel over wireless connectivity between the communication device and a remote destination over a network. Via second signaling associated with the communication session protocol, the mobile communication device transmits non-voice communications (such as biometric data) associated with the user over the wireless communication link from the communication device to the remote destination.

### BACKGROUND

Conventional communication devices can be configured to receive monitor information from a monitor device such as worn by a user operating a respective mobile communication device. The respective mobile communication device can be controlled to display the monitor information. Via the displayed monitor information, the user is able determine whether the user has a health issue.

### BRIEF DESCRIPTION OF EMBODIMENTS

Embodiments herein provide improved implementation of wireless access networks and expanded use of a mobile communication device to provide monitor information to an entity such as a care manager or caregiver.

More specifically, a wireless network environment as discussed herein includes a mobile communication device (a.k.a., user equipment), a wireless network, and a data management resource. In response to receiving input from the user operating the communication device, via first signaling associated with a communication session protocol, the mobile communication device establishes a voice channel over wireless connectivity (wireless connection) between the communication device and a remote destination over a network. Via second signaling associated with the communication session protocol, the mobile communication device transmits non-voice communications (such as including biometric monitor data) associated with the user over the wireless communication link from the communication device to the remote destination.

In further example embodiments, the non-voice communications communicated over the wireless communication link include biometric data associated with a biometric parameter of the user as monitored by the communication device. The communication device operated by the user can be configured to receive the biometric data from any suitable resource. In one embodiment, the communication device receives the biometric data from a wearable health monitor device operative to monitor one or more biometric parameters associated with the user. In one embodiment, the biometric data includes historical data stored by the communication device prior to establishing the voice channel.

In an example embodiment, the biometric data is near real-time data collected by the communication device after establishment of the voice channel. The communication device can be configured to stream the biometric data such as to a care manager as the biometric data is collected by the communication device from the wearable health monitor device.

Further embodiments herein include, via the communication device, communicating the non-voice communications (such as biometric data) associated with the user from the communication device to the remote destination (such as care management resource or caregiver) in response to a message received from an entity at the remote destination requesting medical information from the user. In one embodiment, if the operator permits access and allows the operator at the remote destination to take control of the wearable monitored device and control flow of biometric data from the mobile communication device (as collected from the wearable monitor device) to the remote destination, the message(s) from the remote destination include one or more commands to retrieve biometric data associated with the wearable monitor device. The communications may allow the remote operator to control operation of the wearable monitor device (i.e., biometric monitor device). Thus, it is possible that the monitor device is not initially configured to monitor biometric data of a particular type. The remote operator taking over control of the monitored device may configure it to collect biometric data of a particular type for forwarding through the communication device to the remote destination. Thus, commands can be communicated from a caregiver or care manager over the network through the mobile communication device operated by the user to the wearable monitor device. The wearable monitor device provides biometric data as indicated by the received commands from the caregiver and/or care manager.

In still further example embodiments, in response to detecting that the user of the communication device would like to communicate or at least allow conveyance of biometric data to the remote destination, the mobile communication device i) retrieves biometric data associated with the user (locally in the communication device or from the wearable monitor device), and ii) communicates the retrieved biometric data over the established wireless connectivity to the remote destination.

It may be desirable to establish a secure wireless communication link between the mobile communication device and the remote destination such that the biometric data is not used by unauthorized parties. For example, the remote destination may provide notification to the mobile communication device of a public key associated with the remote destination. In one embodiment, the remote destination provides a respective source identifier (such as specified by a network address or URL) indicating a source and/or corresponding location from which the mobile communication device is to retrieve the public key. The mobile communication device retrieves or receives the public encryption key associated with the remote destination (such as caregiver or care manager). Via the public key, the mobile communication device encrypts the non-voice communications (such as communications including monitored biometric parameters) associated with user. The mobile communication device communicates the encrypted non-voice communications over the wireless communication link in accordance with the communication session protocol to the remote destination.

Thus, the communication device can be configured to receive monitor data from a biometric monitoring device (such as wearable monitor device) worn by the user; the communication device communicates the monitor data in the non-voice communications from the communication device over the wireless communication link to the remote destination.

In still further example embodiments, the user of the communication device generates the input (such as a command) to establish the voice channel in response to the user seeking medical assistance from a caregiver. More specifically, the user of the communication device seeks medical assistance from the caregiver or care management resource.

Still further example embodiments herein include, via signaling (such as transmission and receipt of messages) associated with the communication session protocol, the mobile communication device can be configured to receive a network address indicating a source from which to retrieve an encryption key. The mobile communication device retrieves the encryption key via communications to the source. The mobile communication device then encrypts the non-voice communications via the encryption key.

As previously discussed, the non-voice communications may include monitor data generated by a biometric monitor device worn by the user. In further example embodiments, the notification application or other suitable entity as discussed herein tracks a history of biometric data generated by the monitor device and that is communicated from the communication device to respective remote caregiver entities with respect to the communication device.

Yet further embodiments herein include, via the notification application or other suitable entity, temporarily revoking or preventing distribution of the monitor data to remote entities for one or more time durations (timeslots, time period, etc.) as indicated by a schedule selected by the user.

Embodiments herein are useful over conventional techniques. For example, implementation of communication management resource in a mobile communication device enables useful conveyance of information such as biometric data from a mobile communication device to a remote destination. If desired, the remote destination is able to forward the received biometric data to a target destination such as a caregiver who uses the biometric data as a basis of determining how to provide appropriate care. Alternatively, the caregiver receives the biometric data provided by he wearable monitor device.

Note that any of the resources as discussed herein can include one or more computerized devices, mobile communication devices, sensors, servers, base stations, wireless communication equipment, communication management systems, controllers, workstations, user equipment, handheld or laptop computers, or the like to carry out and/or support any or all of the method operations disclosed herein. In other words, one or more computerized devices or processors can be programmed and/or configured to operate as explained herein to carry out the different embodiments as described herein.

Yet other embodiments herein include software programs to perform the steps and operations summarized above and disclosed in detail below. One such embodiment comprises a computer program product including a non-transitory computer-readable storage medium (i.e., any computer readable hardware storage medium) on which software instructions are encoded for subsequent execution. The instructions, when executed in a computerized device (hardware) having a processor, program and/or cause the processor (hardware) to perform the operations disclosed herein. Such arrangements are typically provided as software, code, instructions, and/or other data (e.g., data structures) arranged or encoded on a non-transitory computer readable storage medium such as an optical medium (e.g., CD-ROM), floppy disk, hard disk, memory stick, memory device, etc., or other medium such as firmware in one or more ROM, RAM, PROM, etc., or as an Application Specific Integrated Circuit (ASIC), etc. The software or firmware or other such configurations can be installed onto a computerized device to cause the computerized device to perform the techniques explained herein.

Accordingly, embodiments herein are directed to a method, system, computer program product, etc., that supports operations as discussed herein.

One embodiment includes a computer readable storage medium and/or system having instructions stored thereon. The instructions, when executed by the computer processor hardware, cause the computer processor hardware (such as one or more co-located or disparately processor devices or hardware) to: in response to receiving input from a user operating a communication device, implement a communication session protocol at a communication device to establish a voice channel over wireless connectivity between the communication device and a remote destination over a network; and via signaling associated with the communication session protocol, support conveyance of non-voice communications (such as biometric data from a wearable monitor device) associated with the user over the wireless communication link from the communication device to the remote destination.

The ordering of the steps above has been added for clarity sake. Note that any of the processing steps as discussed herein can be performed in any suitable order.

Other embodiments of the present disclosure include software programs and/or respective hardware to perform any of the method embodiment steps and operations summarized above and disclosed in detail below.

It is to be understood that the system, method, apparatus, instructions on computer readable storage media, etc., as discussed herein also can be embodied strictly as a software program, firmware, as a hybrid of software, hardware and/or firmware, or as hardware alone such as within a processor (hardware or software), or within an operating system or a within a software application.

As discussed herein, techniques herein are well suited for use in the field of providing improved wireless connectivity in a network environment. However, it should be noted that embodiments herein are not limited to use in such applications and that the techniques discussed herein are well suited for other applications as well.

Additionally, note that although each of the different features, techniques, configurations, etc., herein may be discussed in different places of this disclosure, it is intended, where suitable, that each of the concepts can optionally be executed independently of each other or in combination with each other. Accordingly, the one or more present inventions as described herein can be embodied and viewed in many different ways.

Also, note that this preliminary discussion of embodiments herein (BRIEF DESCRIPTION OF EMBODIMENTS) purposefully does not specify every embodiment and/or incrementally novel aspect of the present disclosure or claimed invention(s). Instead, this brief description only presents general embodiments and corresponding points of novelty over conventional techniques. For additional details and/or possible perspectives (permutations) of the invention(s), the reader is directed to the Detailed Description section (which is a summary of embodiments) and corresponding figures of the present disclosure as further discussed below.

### BRIEF DESCRIPTION OF THE DRAWINGS

FIG. 1 is an example diagram illustrating a communication system including a user wearing a wearable monitor device and distribution management of collected biometric data according to embodiments herein.

FIG. 2 is an example diagram illustrating implementation of a graphical user interface enabling user input according to embodiments herein.

FIG. 3 is an example diagram illustrating a technique of providing supplemental biometric data to a caregiver according to embodiments herein.

FIG. 4 is an example diagram illustrating a flow diagram of a communication session protocol that establishes a voice communication channel and further provides conveyance of supplemental biometric data associated with a user according to embodiments herein.

FIG. 5 is an example diagram illustrating an established voice channel in which a user of a mobile communication device communicates with a caregiver or caregiver management resource according to embodiments herein.

FIG. 6 is an example diagram illustrating a request from a user operating the mobile communication device to provide supplemental biometric data to one or more caregivers according to embodiments herein.

FIG. 7 is an example diagram illustrating simultaneous use of a voice channel and communication session protocol communications between one or more caregivers and a user of the mobile communication device according to embodiments herein.

FIG. 8 is an example diagram illustrating control of a monitor device worn by a user via a remote caregiver providing control commands according to embodiments herein.

FIG. 9 is an example diagram illustrating example computer hardware and software operable to execute operations according to embodiments herein.

FIG. 10 is an example diagram illustrating a method according to embodiments herein.

The foregoing and other objects, features, and advantages of the invention will be apparent from the following more particular description of preferred embodiments herein, as illustrated in the accompanying drawings in which like reference characters refer to the same parts throughout the different views. The drawings are not necessarily to scale, with emphasis instead being placed upon illustrating the embodiments, principles, concepts, etc.

### DESCRIPTION OF EMBODIMENTS

A network environment includes a mobile communication device (a.k.a., user equipment), a wireless network, and a care management resource. In response to receiving input from the user operating the communication device, via first signaling associated with a communication session protocol, the mobile communication device establishes a voice channel over wireless connectivity between the communication device and a remote destination (such as the care management personnel) over a network. Via second signaling associated with the communication session protocol, the mobile communication device transmits non-voice communications (such as biometric data) associated with the user over the wireless communication link from the communication device to the remote destination. The communication session protocol supports notification from the care management personnel of an ability to receive biometric data associated with the user. The second signaling associated with the communication session protocol (previously used to set up the voice communication link) supports communication of biometric data from the communication device to the care management personnel if the user provides appropriate input to the communication device to do so.

Typically, a telecom network doesn't support communication of medical data over a phone call with a caregiver. Thus, during critical situations where a user needs immediate care, a caregiver or care management personnel may not receive any health/vital information and spend considerable time formulating a diagnosis (such as based on collecting and analyzing appropriate biometric data) instead of providing expedient treatment.

Nowadays, many people carry a smart device with them all of the time. In addition to carrying a phone, a user may also wear a biometric monitoring device to monitor biometric parameters such as ECG information, heart rate information, blood oxygen saturation level, body temperature, blood pressure, etc.

As further discussed herein, when a mobile communication device user makes a call to a caregiver or care management personnel, the caregiver or care management personnel may communicate a request for biometric data associated with the user using a SIP:INFO method such as so-called mid call signaling in a communication session protocol used to initially establish a voice communication link. For example, in one embodiment, a SIP:INFO message such as associated with a signaling path already established during a voice call setup notifies a mobile communication device of a Public key for encryption. The end user operating the mobile communication device is prompted via a message on a display screen of the mobile communication device regarding an ability to communicate biometric data associated with the user to the caregiver or care management personnel.

Assume that the user provides input to the communication device and/or wearable monitor device to share health data (such as biometric data) with the caregiver or care management personnel. In such an instance, in response to a decision to share biometric data, a collection application on the mobile communication device retrieves biometric data associated with the user (such as received from a monitor device worn by the user or previously stored biometric data) and encrypts it using the public key (such as downloaded using a URL received in SIP:INFO method). After verifying the certificate validity (public key) with a certificate authority, the communication device includes the encrypted biometric data associated with the user in a body of SIP:INFO method (message or function) and communicates it to the caregiver and/or care management personnel. The caregiver or care management personnel uses the received biometric data to provide care to the user. The care may include providing voice feedback over the communication link to the user operating the communication device, controlling the wearable monitor device, etc. For example, the caregiver and/or care management personnel may also determine the need for specific biometric data associated with the user. In one embodiment, the user of the mobile communication device allows the caregiver and/or care management personnel to control operation of the monitor device worn by the user to retrieve biometric parameters of interest.

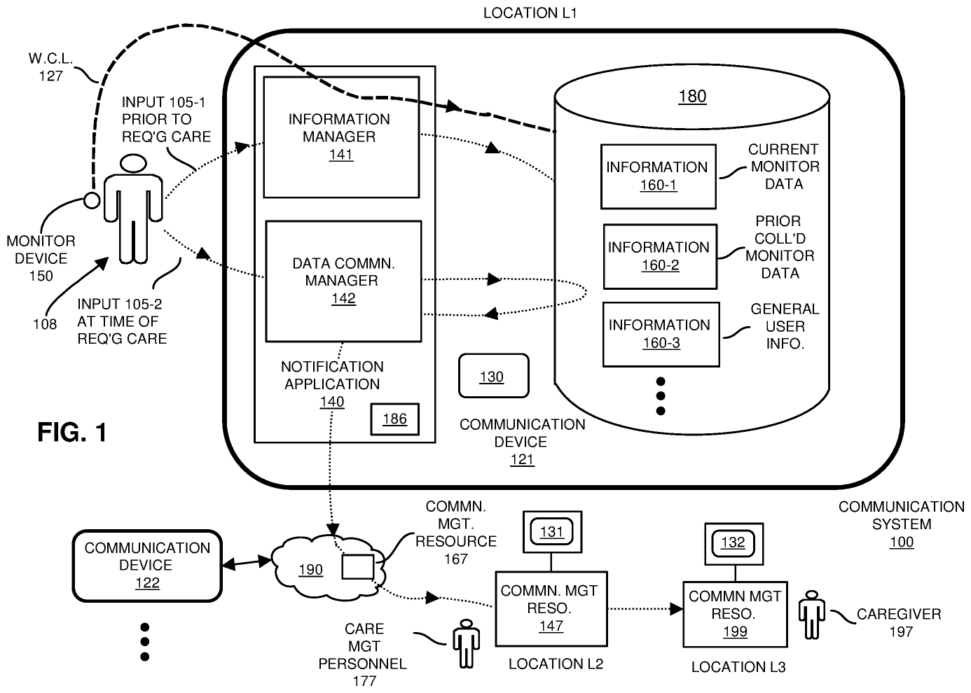

FIG. 1 is an example diagram of a communication system for managing medical care notifications according to embodiments herein.

As shown, communication system 100 includes communication devices 121, 122, etc. The communication devices communicate over network 190 with persons, resources, etc., at a remote location such as care management personnel 177 and/or caregiver 197. By way of a non-limiting example, each of the communication devices 121, 122, etc., can be any suitable type of device such as a cellular phone device, user equipment, mobile device, personal digital assistant, touch pad device, portable computer, wire-line telephone, wireless phone, wireless mobile device, etc.

The communication device 121 in this example embodiment includes information manager 141, data communication manager 142, notification application, etc., to facilitate different functions as discussed herein. Note that each of the functions can be implemented in any suitable manner. For example, the information manager 141 can be implemented as information manager hardware, information manager software, or a combination of information manager hardware and information manager software; the data communication manager 142 can be implemented as a communication manager hardware, data communication manager software, or a combination of data communication manager hardware and data communication manager software: notification application 140 can be implemented as notification hardware, notification software, or a combination of notification hardware and notification software; and so on.

Network 190 can be and/or include any suitable type of network supporting communications between communication devices 121, 122, etc., and or other resources in communication system 100. For example, network 190 can be and/or include a phone network, wireless network, wired network, cellular phone network, Internet, local area network, public switched telephone network, etc.

The following textual description describes functionality supported by communication device 121 and communications system 100. Note that each of the other communication devices in communication system 100 can provide similar functionality for each of the different users.

In one embodiment, the user 108 of the communication device 121 downloads notification application 140 onto his/her communication device 121. The application can be retrieved from a remote resource over network 190.

As shown, notification application 140 can include functionality such as information manager 141, data communication manager 142, etc.

Subsequent to installation of the notification application 140, and prior to occurrence of a request for medical care, the user 108 stores personal information 160-3 (such as general user information) in repository 180. Repository 180 can be located in the communication device 121 or located at a remote location with respect to the communication device 121.

Note that the information 160 can include different types of information for each of multiple different types of possible future notifications. For example, information 160-1 includes information such as current or most recently received monitor data from the monitor device 150 (such as wearable monitor device) that may be useful in the event of a first type of circumstance; information 160-2 includes information such as prior collected monitor data from the monitor device 150 that may be useful in the event of a second type of circumstance; information 160-3 includes general information associated with the user 108; and so on.

In one embodiment, the user 108 wears a monitor device 150 that monitors one or more parameters associated with the user 108 such as pulse rate, blood pressure, body temperature, blood oxygen saturation level, etc. The communication system 100 includes a wireless communication link 127 between the monitor device 150 and the communication device 121. The wireless communication link 127 conveys the monitor data collected by the monitor device 150 and stores it as information 160-1 and information 160-2. Information 160-1 represents most recent collected monitor data; information 160-2 represents historical collected data.

Subsequent to storing different types of information 160 in repository 180, assume that the user 108 would like to generate a notification seeking care about a medical condition. In such an instance, via input 105-2, the user 108 of the communication device 121 initiates execution of the data communication manager 142 in notification application 140.

As further shown, assume that the user 108 requests care from a remote caregiver such as care management personnel 177 and/or caregiver 197. In one embodiment, as further discussed herein, the user supplies input 105-2 requesting care from care management personnel 177 and/or caregiver 197. In such an instance, the notification application 140 establishes connectivity with one or more remote entities such as care management personnel 177 and/or caregiver 197. The notification application 140 communicates any of the information (such as information 160-1, information 160-2, information 160-3, etc.) stored in repository 180 to the care management resource 147 (such as phone, computer, etc.) and care management personnel 177 and/or communication management resource 199 (such as phone, computer, etc.) and caregiver 197.

In one embodiment, the notification application 140 and/or communication device 121 includes or generates tracking information 186. Via the tracking information 186, the notification application 140 or other suitable entity tracks a history of biometric data generated by the monitor device 150 and that is communicated from the communication device 121 to respective one or more remote caregiver entities (such as care management personnel 177, caregiver 197, etc.) with respect to the communication device 121. The tracking information 186 can indicate a respective time and date of the communication device 121 transmitting specific biometric data generated by the monitor device 150. More specifically, in one embodiment, for each of multiple instance of communicating biometric data, the tracking information 186 can indicate the type of biometric data communicated to a particular recipient as well as a corresponding time of communicating the biometric data to the recipient. Thus, embodiments herein include maintaining a history with whom the user 108 previously shared data and what type of monitored data as well as when the biometric data was shared. This provides the user 108 the ability to do a privacy audit at a later time.

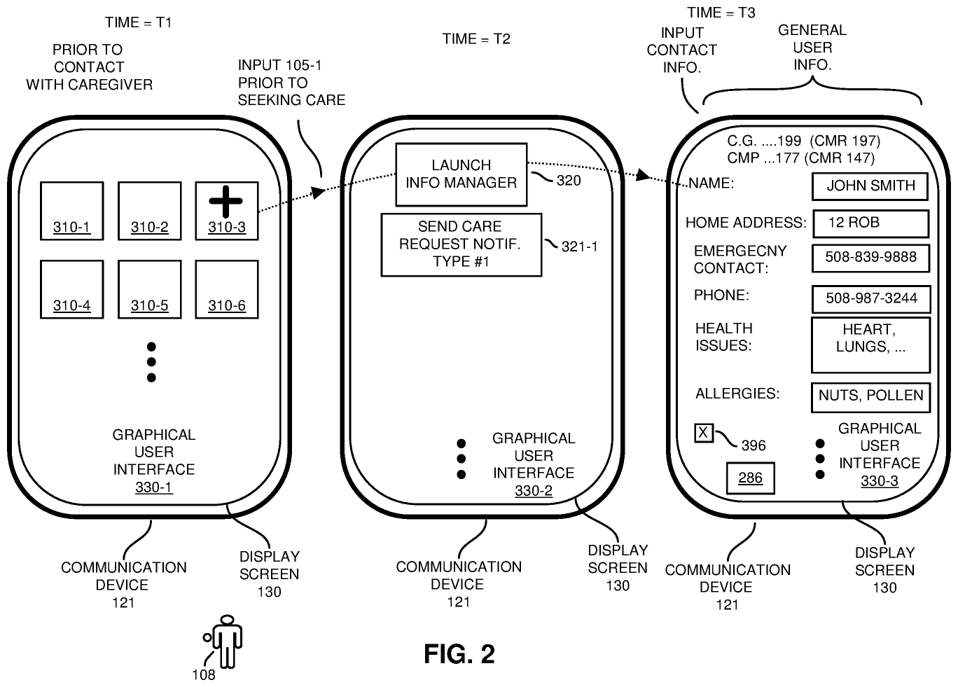

FIG. 2 is an example diagram illustrating an application for inputting information for each of multiple different types of emergencies according to embodiments herein.

For example, as shown, communication device 121 includes a display screen 130. Display screen 130 initially displays graphical user interface 330-1 indicating, to the user 108, different downloaded applications. Each of the icons 310 (e.g., icon 310-1, icon 310-2, icon 310-3, icon 310-4, icon 310-5, icon 310-6, and so on) indicates a different selectable application.

In one embodiment, display of the graphical user interface 330 at a particular time can be contingent upon the user 108 providing the appropriate authorization information (e.g., username, password, etc.) to operate the communication device 121. In certain embodiments as further discussed below, the user 108 can view the icon 310-3 and/or execute notification application 140 even before input of the appropriate authorization information.

User selection of icon 310-1 enables the user 108 to execute a first application, selection of icon 310-2 enables the user 108 to execute a second application, selection of icon 310-3 enables the user 108 to execute a third application, and so on.

In this example, selection of icon 310-3 enables the user 108 to initiate execution of the notification application 140. More specifically, prior to occurrence of a request for care, the user selects (e.g., via tapping, clicking, speaking, etc.) icon 310-3. Selection of icon 310-3 in graphical user interface 330-1 at time T1 causes the notification application 140 to be launched by the communication device 121.

As further shown at time T2, launching of the notification application 140 causes the communication device 121 to display graphical user interface 330-2 on display screen 130 of the communication device 121. Graphical user interface 330-2 includes multiple selectable icons 320 and 321-1. The user 108 can select amongst the different icons on graphical user interface 330-2 in order to perform different functions.

In this example, the user 108 selects icon 320 to launch the information manager 141 to input general information about the user 108. In response to receiving such a selection, the information manager 141 initiates display of graphical user interface 330-3 at time T3. As shown, the graphical user interface 330-3 on display screen 330 enables the user to input information 160. For example, the graphical user interface 330-3 includes prompts indicating different types of information to be inputted by the user 108 for each of multiple type sequence of frames possible future circumstances. The user 108 inputs information such as text, files, multi-media, links, etc., in appropriate data fields.

Note that in addition to the data fields, the graphical user interface 330-3 can include boxes in which the user 108 can check ON or OFF to selectively perform different standard functions. For example, the graphical user interface 330-3 can include a location tracker box 396 associated with transmission of location information in the event of a desire to allow tracking by care management personnel and/or caregiver 197. When the location information box 396 is checked by the user 108 during setup, the communication device 121 can be configured to retrieve location information during a request for care and forward it to the care management personnel and/or caregiver 177. When the box 396 is not checked, the communication device 121 will not retrieve location information during the reported care request for forwarding to the care management personnel and/or caregiver 177.

In one embodiment, the communication device 121 can be configured to enable access to the notification application 140 and/or respective icon 310-3 even though the communication device 121 may otherwise be locked. For example, when initially powering the communication device or activating it for use, the communication device 121 can be configured to display a request on the display screen 330 for the user to enter a PIN (i.e., Personal Identification Number) or password to unlock the communication device 121 for full use. Certain applications such as the notification application 140 may not require entry of the authorization information before use. Accordingly, in one embodiment, the user 108 of the communication device 121 may be able to view the icon 310-3 and/or execute the notification application 140 and its functionality without having to provide the authorization information (e.g., a password, PIN, etc.). In such an embodiment, the user 108 of the communication device 121 can make a care request call and/or view contact information data stored on the phone without being slowed down by authorization.

If desired, the user 108 can configure the general user information to further include contact information such as an identity of the caregiver 199 associated with the communication management resource 197 as well as the care management personnel 177 associated with the communication management resource 147. This information can include any suitable information such as phone number, network address, etc., indicating how to contact and/or provide connectivity between the communication device 121 and those different entities (caregiver 199, communication management resource 197, care management personnel 177, communication management resource 147, etc.).

In further example embodiments, via display region 286 (such as including display of a calendar, data fields, etc.) in which to input data/commands, the graphical user interface 330-3 provides the user 108 an opportunity to restrict distribution of collected biometric data (as generated by the monitor device 150) in one or more different time durations as specified by schedule information. More specifically, via the display region 286, the user can input schedule to times and distribution of different types of biometric data collected by the monitor device 150. In one embodiment, in accordance with schedule information provided (such as display region 286) by the user 108, the notification application 140 or other suitable entity temporarily revokes or prevents distribution of the biometric monitor data with remote entities for one or more time durations as indicated by a schedule selected by the user (as inputted via display region 286). Thus, embodiments herein include revocation of data sharing authorization or time-based data sharing such as for a few hours or days, or customization of distribution periods with options to select what monitored data is to be share or not shared at such times.

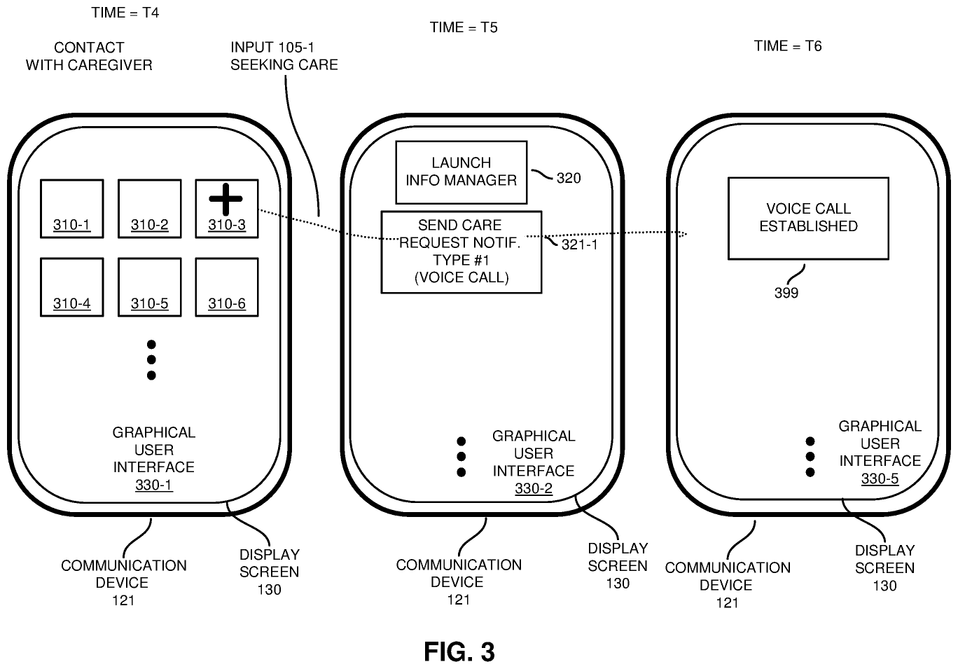

FIG. 3 is an example diagram illustrating a technique of providing supplemental biometric data to a caregiver according to embodiments herein.

As previously discussed, subsequent to submitting information 160-3, the monitor device 150 worn or operated by the user 108 monitors one or more parameters of the user 108 and communicates the corresponding data over wireless communication link 127 to the mobile communication device for storage as information 160-1 and information 160-2.

Assume in this example embodiment that a medical condition occurs and the user 108 or notification application 140 decides to seek medical care. The request for medical care may occur in response to detecting that biometric data from the wearable monitor device indicates a medical condition needing attention. Thus, in one embodiment, the notification application 140 can be configured to monitor one or more parameters and seek medical care on behalf of the user 108 in response to detecting a medical condition such as that one or more parameters monitored by the monitor device 150 exceed or fall below a normal a threshold level for that parameter. Prior to sending a care request, the notification may generate an audible or visual notification that the medical condition was detected and ask the user 108 whether to seek medical attention.

In response to this condition, in a manner as previously discussed, assume that the user 108 or notification application 140 selects icon 310-3 prompting display of the graphical user interface 330-2 on the display screen 130.

To request medical assistance from a remote caregiver, the user 108 further selects function 321-1 via touching the corresponding symbol displayed on the display screen 130. This selection prompts the notification application 140 of the mobile communication device 121 to establish a voice call with one or more care service providers such as care management personnel 177 and/or caregiver 197.

After the voice call connection is established with the care management personnel 177 and/or caregiver 197, the notification application 140 displays graphical user interface 330-5 on the display screen 130 indicating the established voice connection via a respective visual indication 399. Via the voice connection, the user 108 is able to communicate with the care management personnel 177 and/or caregiver 197.

As further discussed herein, in response to receiving input 105-2 from the user operating the communication device, via first signaling associated with a communication session protocol, the mobile communication device establishes a voice channel over wireless connectivity between the communication device and a remote destination over a network. Via second signaling associated with the communication session protocol, the mobile communication device transmits non-voice communications associated with the user over the wireless communication link from the communication device to the remote destination.

Additional details of establishing voice connectivity and providing supplemental biometric data from the monitor device 150 is further discussed below.

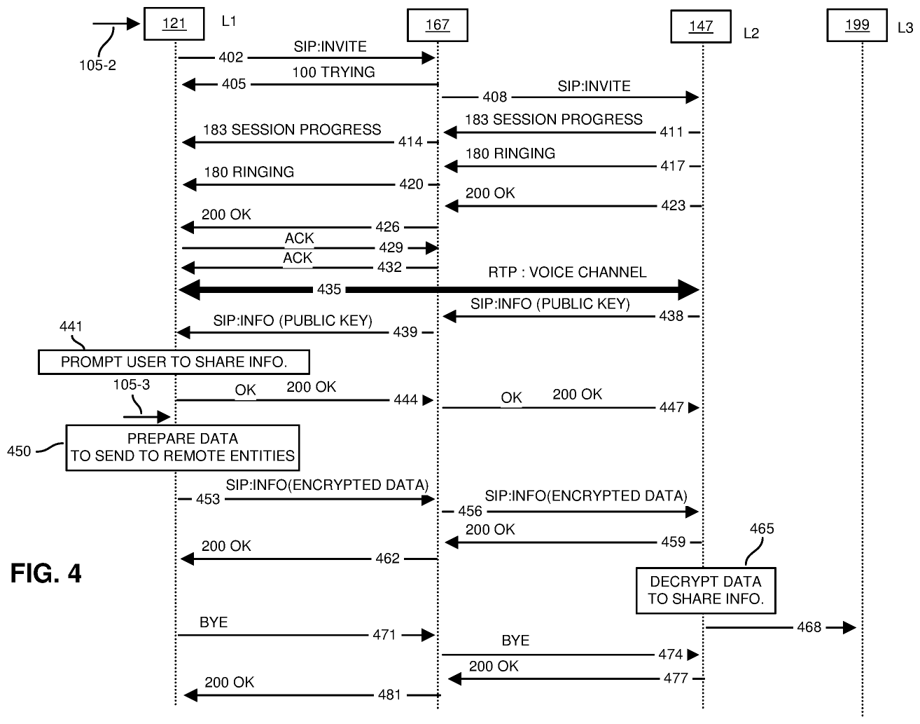

FIG. 4 is an example diagram illustrating a flow diagram of a communication session protocol that establishes a voice communication channel and further provides conveyance of supplemental biometric data associated with a user according to embodiments herein.

In one embodiment, the communication device 121 and corresponding resources in network 190 support establishing connectivity between the mobile communication device 121 and the care management personnel 177 and/or caregiver 197 via a so-called communication session protocol or, a.k.a., SIP. The Session Initiation Protocol (SIP) is a signaling protocol used for initiating, maintaining, and terminating real-time sessions that include voice, video and messaging applications. SIP is used for signaling and controlling multimedia communication sessions in applications of Internet telephony for voice and video calls, in private IP telephone systems, in instant messaging over Internet Protocol (IP) networks as well as mobile phone calling over LTE (VoLTE).

The SIP protocol defines the specific format of messages exchanged and the sequence of communications for cooperation of the participants. SIP is a text-based protocol, incorporating many elements of the Hypertext Transfer Protocol (HTTP) and the Simple Mail Transfer Protocol (SMTP).

As shown, in response to receiving the input 105-2 to contact the care management personnel 177 and/or caregiver 197, via communications 402, the notification application 140 of the communication device 121 establishes connectivity to the care management personnel 177 at communication management resource 147 over a wireless communication link. In one embodiment, the user 108 of the communication device 121 generates the input 105-2 (such as a command) to establish the voice channel supporting communications 435 in response to the user seeking medical assistance from a caregiver.

More specifically, to establish a respective voice call connection between the communication device 121 and the care management personnel 177, the notification application 140 (i.e., communication device 121) implements a so-called communication session protocol such as Session Initiation Protocol or SIP and, via same, transmits communications 402 such as an SIP:INVITE message from the notification application 140 to communication management resource 167 (call/data management resource) in the 190.

In response to receiving the communications 402, the communication management resource 167 transmits communications 405 such as indicating that the communication management resource 167 is attempting to establish a voice call with the care management personnel 177 at a remote destination (such as location L2).

As further shown, via communications 408, the communication management resource 167 transmits a respective SIP:INVITE message to the care management personnel 177.

In response to receiving the communications 408, the care management personnel 177 transmits a respective session progress message (183) via communications 411 to the communication management resource 167. In one embodiment, the 183 Session Progress message indicates whether the calling user agent should provide alerting or not. Further, in response to receiving communications 411, the communication management resource 167 associated with network 190 transmits communications 414 such as a session progress message (183) to the communication device 121.

Via communications 417 (such as 180 ringing), the care management personnel 177 notifies the communication management resource 167 that the call alert (associated with the INVITE) is received by the care management personnel 177 and communication management resource 147. Via communications 420 (such as 180 ringing), the communication management resource 167 notifies the communication device 121 that the call alert (associated with the INVITE) is received by the communication management resource 147 operated by the care management personnel 177.

Via communications 423 (such as 200 OK), the care management personnel 177 notifies the communication management resource 167 that the call request was received and understood and is being processed by the communication management resource 147. Via communications 426 (such as 200 OK), the communication management resource 167 relays the 200 OK message to the communication device 121.

Via communications 429, the communication device 121 communicates an acknowledgement message to the communication management resource 167. Via. communications 431, the communication management resource 167 communicates an acknowledgement message to the notification application 140 of the communication device 121.

The result of implementing the prior communications in the channel selection information protocol is establishment of a respective voice call connection (such as via communications such as an RTP call connection voice channel) between the mobile communication device 121 and the care management personnel 177, The voice channel 435 supports voice communications between the user 108 operating the mobile communication device 121 at location L1 and the care management personnel 177 at location L2.

As further shown, the communication session protocol used to establish the voice call supports conveyance of supplemental data (such as biometric data stored in information 160-1 and information 160-2 provided by the mobile device 150) to the care management personnel 177 and/or caregiver 197.

More specifically, via communications 438, the care management personnel 177 can be configured to communicate public key information (actual public key or URL link to retrieve the public key information) associated with the care management personnel 177 and/or caregiver 197. Via communications 439, the communication management resource communicates public key information (actual public key or URL link to retrieve the public key information) associated with the care management personnel 177 and/or caregiver 197 to the communication device 121. In one embodiment, the public key information associated with the care management personnel 177 and/or caregiver 197 enables the notification application 140 of the mobile communication device 121 to encrypt data (such as biometric data stored in information 160-1 and 160-2) as further discussed herein.

As further shown, in response to establishing the voice communication connection (such as communication link supporting bidirectional voice communications 235) between the mobile communication device 121 and the care management personnel 177, in processing operation 441, the notification application 140 of the communication device 121 prompts (such as via visual indication on the display screen 130) the user 108 whether it is permissible for the notification application 140 and mobile communication device 121 to share information collected by the monitor device 150 to the care management personnel 177 and/or caregiver 197.

Thus, example embodiments herein include, via signaling (such as transmission and receipt of messages) associated with the communication session protocol such as communications 438 and 439, the mobile communication device 121 can be configured to receive a network address, URL, etc., of a source from which to retrieve an encryption key associated with the communication management resource 147 and/or communication management resource 199. The mobile communication device 121 can be configured to retrieve the encryption key via communications to the source based on the network address. The mobile communication device 121 then encrypts the non-voice communications (such as biometric data in information 160-1 and 160-2) via the encryption key in processing operation 441.

Via communications 444 (such as 200 OK), the communication device 121 notifies the communication management resource 167 that the public key information was received and the corresponding option of providing supplemental is being processed. by the communication device 121. Via communications 447 (such as 200 OK), the communication management resource 167 relays the 200 OK message to the care management personnel 177 and/or caregiver 197.

In response to display of a visual indication on display screen 130, assume that the user 108 provides input 105-3 to communicate the supplemental data (such as biometric data produced by the monitor device 150) to the care management personnel 177 and/or caregiver 197. In response to the input 105-3, in processing operation 450 the notification application 140 prepares biometric data (information 160-1, 160-2, 160-3, etc.) to send to the care management personnel 177 and/or caregiver 197. The processing operation can include encrypting the biometric data with the provided encryption key.

Via communications 453, the notification application of the communication device 121 communicates the encrypted biometric data (one or more of information 160-1, 160-2, 160-3, etc.) and communicates such information to the communication management resource 167. In one embodiment, the communication device 121 communicates the communications 453 such as encrypted biometric data in a SIP:INFO message via the communication session protocol to the communication management resource 167. As further shown, the communication management resource 167 further communicates the received encrypted data in communications 456 to the communication management resource 147 operated by the care management personnel 177 and/or communication management resource 199 operated by the caregiver 197. In one embodiment, the communication management resource 167 communicates the communications 456 such as encrypted biometric data in a SIP:INFO message via the communication session protocol (such as SIP) to the care management personnel 177 and/or caregiver 197.

Via communications 459 (such as 200 OK), the communication device 121 notifies the communication management resource 167 that the encrypted biometric data was received and being processed by the care management personnel 177 and/or caregiver 197. Via communications 462 (such as 200 OK), the communication management resource 167 relays the 200 OK message to the communication device 121.

In processing operation 465, the care management personnel 177 decrypts the received data in communications 456 and communicates the decrypted information (such as one or more of information 160-1, 160-2, and 160-3) via communications 468 to the communication management resource 199 and corresponding caregiver 197. In one embodiment, the care management personnel 177 communicates the biometric data (such as one or more of information 160-1, 160-2, and 160-3) associated with the user 108 to the caregiver 197. The user 108 and the caregiver 197 may reside at the same location. The caregiver 197 may be in motion to visit the user 108. Alternatively, the user 108 may be in transit to meet the caregiver.

Eventually, the user 108 may end the established voice call supporting communications 435. In such an instance, via communications 471, the communication device 121 communicates a BYE message (such as end the voice all connection and/or the communication session in general) in accordance with the communication session protocol to the communication management resource 167. As further shown, via communications 474, the communication management resource 167 relays the BYE message to the care management personnel 177.

Via communications 477 (such as 200 OK), the care management personnel 177 notifies the communication management. resource 167 that the care management personnel 177 confirms the end session request (BYE message). Via communications 481 (such as 200 OK), the communication management resource 167 relays the 200 OK message to the communication device 121.

Thus, the non-voice communications communicated over the wireless communication link from the communication device 121 to through the communication management resource 167 to the care management personnel 177 via the communication session protocol may include biometric data (such as information 160-1, 160-2) associated with one or more monitored biometric parameters of the user 108 as monitored by the monitor device 150.

As previously discussed, the communication device 121 operated by the user 108 can be configured to receive the biometric data from any suitable resource. In one embodiment, the communication device 121 receives the biometric data from a health monitor device 150 that monitors one or more biometric parameters associated with the user. In one embodiment, the biometric data includes historical data stored by the communication device prior to establishing the voice channel (e.g., voice channel supporting communications 435). In an example embodiment, the biometric data is collected in near real-time to the actual measurements by the monitor device after establishment of the voice channel. For example, the information 160-1 represents the latest collected biometric data from the monitor device 150. The notification application 140 of the communication device 121 can be configured to stream the biometric data received from the monitor device 150 as the biometric data is collected by the communication device 121 from the health monitor device 150.

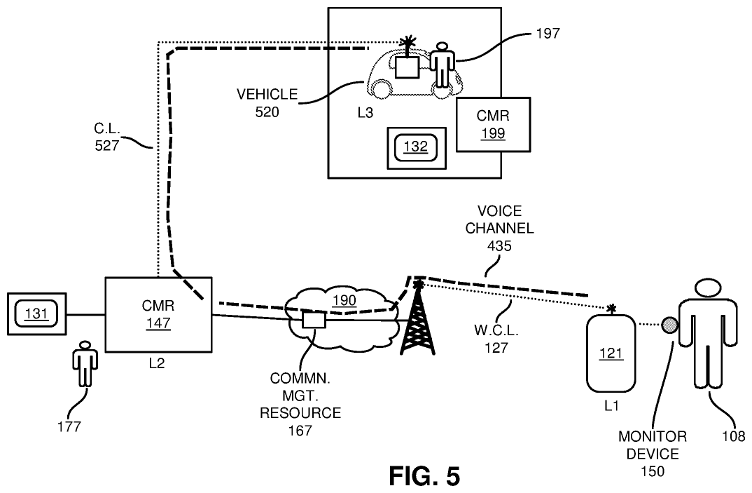

FIG. 5 is an example diagram illustrating an established voice channel according to embodiments herein.

As shown in FIG. 5, the communications system as discussed herein can be configured to establish a respective voice channel between the mobile communication device 121 and the communication management resource 147 and/or communication management resource 199, enabling the user 108 to communicate with care management personnel 177. Note that the voice channel can be extended from the communication management resource 147 to the communication management resource 199 via communication link 527. Alternatively, the communication device 121 can be configured to establish the voice channel 435 via a direction communication link between the mobile communication device 121 and the communication management resource 199 (such as communication device) operated by the caregiver 197.

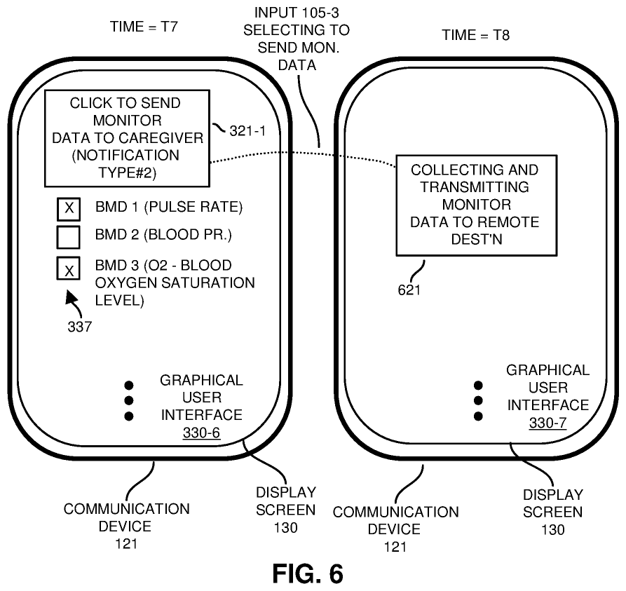

FIG. 6 is an example diagram illustrating a request from a user operating the mobile communication device to provide supplemental biometric data to one or more caregivers according to embodiments herein.

As previously discussed, in response to establishing the voice communication channel 435, the notification application 140 determines that the communication device 121 established a respective communication link with a particular entity such as communication management resource 147 operated by care management personnel 177 and/or communication management resource 199 operated by caregiver 197.

In one embodiment, when the user 108 selects the option 321-1 on graphical user interface 330-6 (which is displayed after establishing the voice channel 435), the notification application 140 displays the option 321-1 on the display screen 130.

In still further example embodiments, in response to detecting that the user of the communication device would like to communicate or at least allow conveyance of the biometric data to the remote destination via selection (tapping) of the option 321-1, in a manner as previously discussed, the mobile communication device 121 displays notification 621 in graphical user interface 330-7 indicating that the mobile communication device 121 is: i) retrieving biometric data associated with the user 108, and ii) communicating it to the retrieved biometric data over the established wireless connectivity to the remote destination.

In one embodiment, the graphical user interface 330-6 can be configured to include different types of monitor data in which to communicate to the target recipient such as caregiver 197 or care management personnel 177. The user 108 can select which types of biometric data to send such as pulse rate, blood pressure, body temperature, blood oxygen saturation level, etc. The user 108 can select the information at any time. For example, the user 108 can select one or more biometric data boxes 337 just after the call (voice channel 235) is established. The user 108 can pre-select one or more biometric data boxes 337 prior to requesting to establish the voice channel 235. The user 108 may provide authorization for the care management personnel 177 and/or caregiver 177 to take control of the graphical user interface 330-6 and select one or more biometric data boxes 337. In such an instance, the notification application 140 collects only the selected biometric data from the wearable monitor device to the target entity.

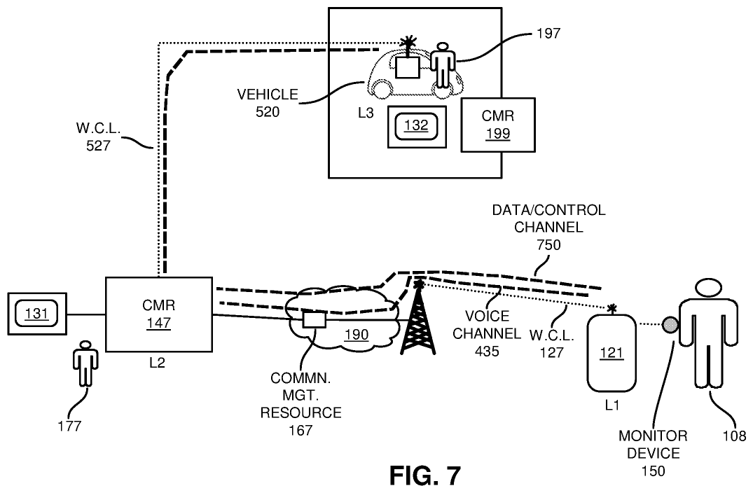

FIG. 7 is an example diagram illustrating simultaneous use of a voice channel and communication session protocol communications between one or more caregivers and a user of the mobile communication device according to embodiments herein.

As previously discussed, the communication system as discussed herein can be configured to establish a respective voice channel to support voice communications between the mobile communication device 121 and the care management personnel 177 and/or the caregiver 197.

Thus, the communication device 121 can be configured to receive monitor data from a biometric monitoring device 150 worn by the user; the communication device 121 further communicates the monitor data in the non-voice communications over the data/control channel from the communication device 121 over the wireless communication link 127 to the remote destination.

As previously discussed, the caregiver 197 and/or care management personnel 177 can be configured to establish a respective data/control channel 750 with the communication device 121 to control operation of the monitor device 150 and retrieve biometric data of interest.

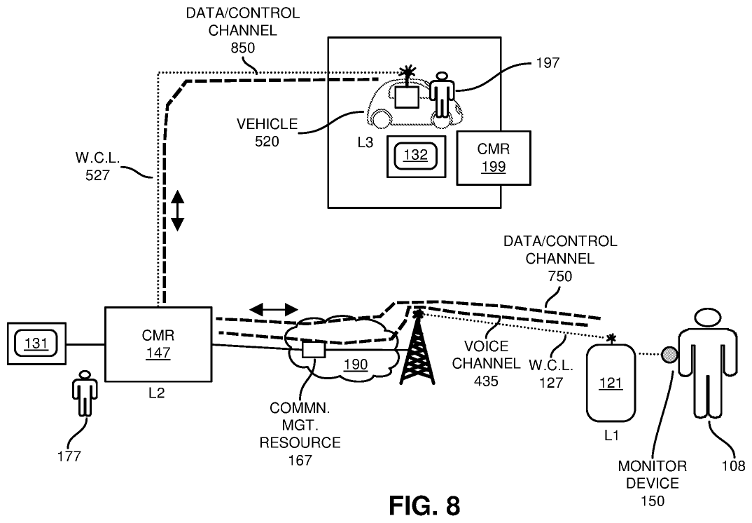

FIG. 8 is an example diagram illustrating control of a monitor device on a user via a remote caregiver according to embodiments herein.

As shown, the user 108 is able to communicate with the care management personnel 177 and/or caregiver 197 via the voice communication channel 435.

As previously discussed, the caregiver 197 and/or care management personnel 177 can be configured to establish a respective data/control channel 750 (in addition to the voice channel 435) with the communication device 121 to control operation of the monitor device 150 and retrieve biometric data of interest.

For example, further embodiments herein include, via the communication device 121, communicating the non-voice communications (such as biometric data) associated with the user 108 from the communication device 121 to the remote destination in response to a message (such as received over the data/control channel 750) received from an entity at the remote destination requesting medical information from the user. In one embodiment, if the user 108 (i.e., operator) permits access and allows the care management personnel or caregiver at the remote destination to control flow of biometric data from the mobile communication device 121 to the remote destination, the message(s) from the remote destination (caregiver 197 or care management personnel 177) include one or more commands to retrieve specific biometric data from the communication device 121 and/or monitor device 150.

In certain instances, the notification application 140 can be configured to continuously monitor the user 108 and corresponding biometric data received from the biometric monitor device 150. The notification application 140 can be configured to analyze the biometric data and automatically connect the mobile communication device 121 to the care management personnel 177 and/or caregiver 197 if the notification application 140 detects a concerning condition associated with the user 108 such as based on biometric value of collected data above or below a threshold value. The caregiver 197 and/or care management personnel 177 may be pre-authorized to communicate with the notification application 140 and retrieve biometric data of interest during such instance.

The communications over the data/control channel 750 may allow the remote operator to control operation of the monitored device. Thus, it is possible that the monitor device 150 is not initially configured to monitor biometric data of a particular type. The remote operator (such as caregiver 197 and/or care management personnel 177) taking over control of the monitor device may configure the communication device 121 and/or monitor device 150 to collect biometric data of a particular type for forwarding through the communication device 121 to the requesting entity.

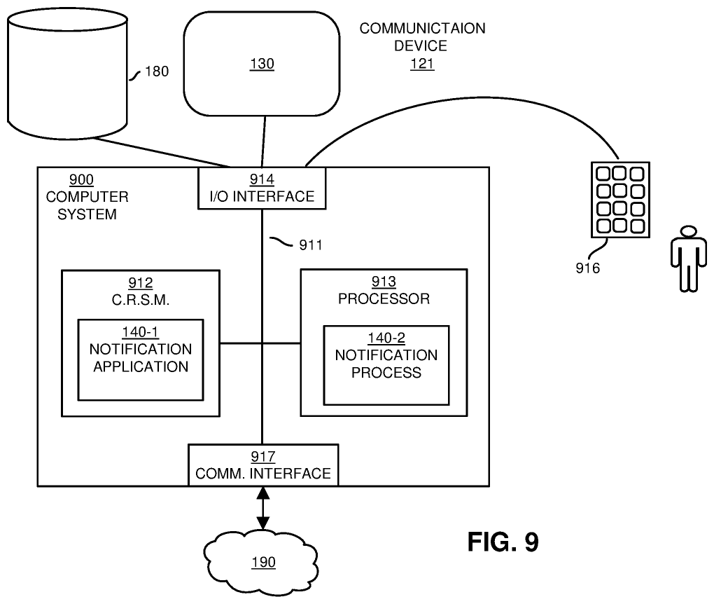

FIG. 9 is an example block diagram of a computer system for implementing any of the operations as previously discussed according to embodiments herein.

Note that any of the resources (such as information manager 141, data communication manager 142, notification manager 140, communication device 121, etc.) as discussed herein can be configured to include computer processor hardware and/or corresponding executable instructions to carry out the different operations as discussed herein.

For example, as shown, computer system 950 of the present example includes interconnect 911 coupling computer readable storage media 912 such as a non-transitory type of media (which can be any suitable type of hardware storage medium in which digital information can be stored and or retrieved), a processor 913 (computer processor hardware), I/O interface 914, and a communications interface 917.

I/O interface(s) 914 supports connectivity to repository 980 and input resource 992.

Computer readable storage medium 912 can be any hardware storage device such as memory, optical storage, hard drive, floppy disk, etc. In one embodiment, the computer readable storage medium 912 is computer-readable storage hardware that stores instructions and/or data.

As shown, computer readable storage media 912 can be encoded with management application 140-1 (e.g., including instructions) in a respective wireless station to carry out any of the operations as discussed herein.

During operation of one embodiment, processor 913 accesses computer readable storage media 912 via the use of interconnect 911 in order to launch, run, execute, interpret or otherwise perform the instructions in management application 140-1 stored on computer readable storage medium 912. Execution of the notification application 140-1 (a.k.a., management application) produces management process 140-2 (a.k.a., management process) to carry out any of the operations and/or processes as discussed herein.

Those skilled in the art will understand that the computer system 950 can include other processes and/or software and hardware components, such as an operating system that controls allocation and use of hardware resources to execute the notification application 140-1.

In accordance with different embodiments, note that computer system may reside in any of various types of devices, including, but not limited to, a mobile computer, a personal computer system, a wireless device, a wireless access point, a base station, phone device, desktop computer, laptop, notebook, netbook computer, mainframe computer system, handheld computer, workstation, network computer, application server, storage device, a consumer electronics device such as a camera, camcorder, set top box, mobile device, video game console, handheld video game device, a peripheral device such as a switch, modem, router, set-top box, content management device, handheld remote control device, any type of computing or electronic device, etc. The computer system 950 may reside at any location or can be included in any suitable resource in any network environment to implement functionality as discussed herein.

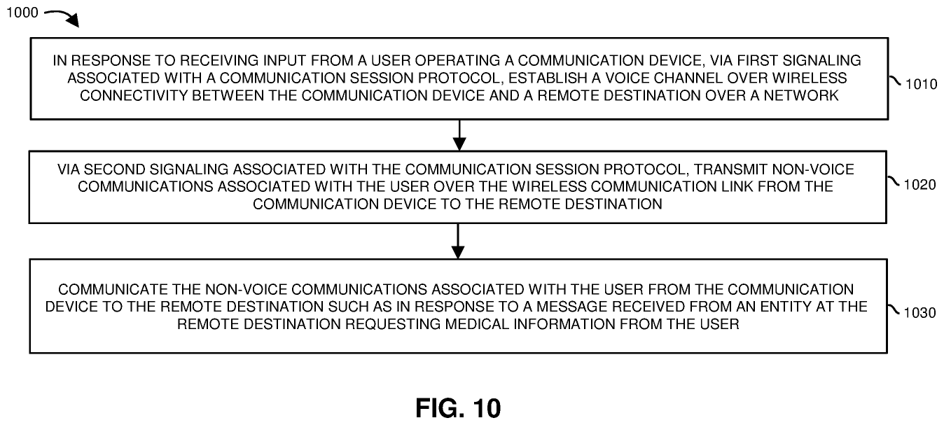

Functionality supported by the different resources will now be discussed via flowcharts in FIG. 10. Note that the steps in the flowcharts below can be executed in any suitable order.

FIG. 10 is a flowchart 1000 illustrating an example method according to embodiments herein. Note that there will be some overlap with respect to concepts as discussed above.

In processing operation 1010, in response to receiving input 105-2 from a user 108 operating communication device 121, via first signaling associated with a communication session protocol implemented by the communication device 121, the communication device 121 establishes a voice channel over wireless connectivity (wireless communication link 127) between the communication device 121 and a remote destination (such as one or more of care management personnel 177 and/or caregiver 197) over one or more networks.

In processing operation 1020, the communication device 121 receives input from the user to communicate non-voice communications associated with the user from the communication device to the remote destination.

In processing operation 1030, via second signaling associated with the communication session protocol (previously used to establish the voice channel separate from the communication session protocol), the communication device 121 transmits non-voice communications (such as biometric data) associated with the user over the wireless communication link from the communication device to the remote destination.

Note again that techniques herein are well suited to facilitate processing of available physical infrastructure information and generation of a proposed wireless network. installation plan for implementation of the new wireless network. However, it should be noted that embodiments herein are not limited to use in such applications and that the techniques discussed herein are well suited for other applications as well.

Based on the description set forth herein, numerous specific details have been set forth to provide a thorough understanding of claimed subject matter. However, it will be understood by those skilled in the art that claimed subject matter may be practiced without these specific details. In other instances, methods, apparatuses, systems, etc., that would be known by one of ordinary skill have not been described in detail so as not to obscure claimed subject matter. Some portions of the detailed description have been presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm as described herein, and generally, is considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has been convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussions utilizing terms such as “processing,” “computing,” “calculating,” “determining” or the like refer to actions or processes of a computing platform, such as a computer or a similar electronic computing device, that manipulates or transforms data represented as physical electronic or magnetic quantities within memories, registers, or other information storage devices, transmission devices, or display devices of the computing platform.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting. Rather, any limitations to the invention are presented in the following claims.

1. A method comprising:
in response to receiving input from a user operating a communication device, via first signaling associated with a communication session protocol, establishing a voice channel over wireless connectivity between the communication device and a remote destination over a network; and
via second signaling associated with the communication session protocol, transmitting non-voice communications associated with the user over the wireless communication link from the communication device to the remote destination.

2. The method as in claim 1, wherein the non-voice communications include biometric data associated with a biometric parameter of the user monitored by the communication device.

3. The method as in claim 2, wherein the biometric data includes historical data stored by the communication device prior to establishing the voice channel.

4. The method as in claim 2, wherein the biometric data is near real-time data collected by the communication device after establishment of the voice channel.

5. The method as in claim 1 further comprising:
communicating the non-voice communications associated with the user from the communication device to the remote destination in response to a message received from an entity at the remote destination requesting medical information from the user.

6. The method as in claim 5, wherein the message is a command from the operator at the remote destination to retrieve the medical information from the communication device.

7. The method as in claim 1 further comprising:
in response to detecting that the user of the communication device would like to communicate data to the remote destination: i) retrieving data associated with the user, and ii) communicating the retrieved data over the established wireless connectivity to the remote destination.

8. The method as in claim 1 further comprising:
receiving a public key associated with the remote destination;
via the public key, encrypting the non-voice communications associated with user; and
communicating the encrypted non-voice communications over the wireless communication link to the remote destination.

9. The method as in claim 1 further comprising:
at the communication device, receiving monitor data from a biometric monitoring device worn by the user; and
communicating the monitor data in the non-voice communications from the communication device over the wireless communication link to the remote destination.

10. The method as in claim 1, wherein the user generates the input to establish the voice channel in response to the user seeking medical assistance from a caregiver.

11. The method as in claim 1 further comprising:
via signaling associated with the communication session protocol, receiving a source associated with retrieval of an encryption key;
retrieving the encryption key via communications from the source; and
wherein the non-voice communications are encrypted via the encryption key.

12. system comprising:
a communication device operative to:
in response to receiving input from a user operating a communication device, implement a communication session protocol to establish a voice channel over wireless connectivity between the communication device and a remote destination over a network; and
via signaling associated with the communication session protocol, support conveyance of non-voice communications associated with the user over the wireless communication link from the communication device to the remote destination.

13. The system as in claim 12, wherein the non-voice communications include biometric data associated with a biometric parameter of the user monitored by the communication device.

14. The system as in claim 13, wherein the biometric data includes historical data stored by the communication device prior to establishing the voice channel.

15. The system as in claim 13, wherein the biometric data is near real-time data collected by the communication device after establishment of the voice channel.

16. The system as in claim 12, wherein the communication device is further operative to:
communicate the non-voice communications associated with the user from the communication device to the remote destination in response to a message received from an entity at the remote destination requesting medical information from the user.

17. The system as in claim 16, wherein the message is a command from the operator at the remote destination to retrieve the medical information from the communication device.

18. The system as in claim 12, wherein the communication device is further operative to:
in response to detecting that the user of the communication device would like to communicate data to the remote destination: i) retrieve data associated with the user, and ii) communicate the retrieved data over the established wireless connectivity to the remote destination.

19. The system as in claim 12, wherein the communication device is further operative to:
receive a public key associated with the remote destination;
via the public key, encrypt the non-voice communications associated with user; and
communicate the encrypted non-voice communications over the wireless communication link to the remote destination.

20. The system as in claim 12, wherein the communication device is further operative to:
at the communication device, receive monitor data from a biometric monitoring device worn by the user; and
communicate the monitor data in the non-voice communications from the communication device over the wireless communication link to the remote destination.

21. The system as in claim 12, wherein the user generates the input to establish the voice channel in response to the user seeking medical assistance from a caregiver.

22. The system as in claim 12, wherein the communication device is further operative to:
via signaling associated with the communication session protocol, receive a network address associated with an encryption key;
retrieve the encryption key via communications to the network address; and
wherein the non-voice communications are encrypted via the encryption key.

23. Computer-readable storage hardware having instructions stored thereon, the instructions, when carried out by computer processor hardware, cause the computer processor hardware to:
in response to receiving input from a user operating a communication device, implement a communication session protocol to establish a voice channel over wireless connectivity between the communication device and a remote destination over a network; and
via signaling associated with the communication session protocol, support conveyance of non-voice communications associated with the user over the wireless communication link from the communication device to the remote destination.

24. The method as in claim 1, wherein the non-voice communications include monitor data generated by a biometric monitor device worn by the user, the method further comprising:
tracking a history of biometric data generated by the monitor device and communicated from the communication device to one or more remote caregiver entities with respect to the communication device.

25. The method as in claim 1, wherein the non-voice communications include biometric data generated by a biometric monitor device worn by the user, the method further comprising:
temporarily preventing distribution of the biometric data with remote entities as specified by a schedule selected by the user.
